(19) United States
(12) Patent Application Publication
(10) Pub. No.: US 2006/0063768 A1
(43) Pub. Date: Mar. 23, 2006

(54) Use of folates for producing a preparation suitable for preventing and treating inflammation and diseases associated with inflammation, especially for influencing the inflammation markers crp and saa

(76) Inventors: Thomas Mueller, Edmonton (CA); Rudolf Moser, Schaffhausen (CH); Martin Ulmann, Dachsen (CH)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/505,777

(22) PCT Filed: Feb. 24, 2003
(86) PCT No.: PCT/EP03/01848

§ 371 Date: Oct. 4, 2005

(30) Foreign Application Priority Data
Feb. 26, 2002 (CH) 337/02

(51) Int. Cl. A61K 31/525

(52) U.S. Cl. 514/251

(57) ABSTRACT

This invention relates to the use of folates for producing a pharmaceutical preparation suitable for the prevention and treatment of inflammation and of diseases associated with inflammation, particularly for influencing the inflammation markers C-reactive protein (CRP) and serum amyloid A protein (SAA). The clinical areas of application are all anomalies of the CRP and SM levels. The invention also relates to pharmaceutical preparations for the prevention and treatment of inflammation and of diseases associated with inflammation, particularly for influencing CRP and SM levels, characterised in that as an active ingredient it comprises at least one compound which is selected from the group consisting of pteroic acid monoglutamate (folic acid), dihydrofolic acid, 5-formyltetrahydrofolic acid, 5-methyltetrahydrofolic acid, 5,10-methylenetetrahydrofolic acid, 5,10-methenyl-tetrahydrofolic acid, 10-formyltetrahydrofolic acid or tetrahydrofolic acid, polyglutamates thereof, optical isomers thereof, particularly optically pure natural isomers thereof, and mixtures of optical isomers also, particularly racemic mixtures, as well as pharmaceutically acceptable salts thereof also, together with pharmaceutically acceptable active ingredients and adjuvants.

This invention relates to the use of folates for producing a preparation suitable for the prevention and treatment of inflammation and diseases associated with inflammation, particularly for influencing the levels of the inflammation markers C-reactive protein (CRP) and serum amyloid A protein (SM). The areas of application are all anomalies of the CRP and SAA levels.

In the present text, the term “folate” relates both to pteroic acid monoglutamate (folic acid) and to reduced forms such as dihydrofolates and tetrahydrofolates, e.g. 5-formyltetrahydrofolic acid, 5-methyltetrahydrofolic acid, 5,10-methylene-tetrahydrofolic acid, 5,10-methenyltetrahydrofolic acid, 10-formyltetrahydrofolic acid and tetrahydrofolic acid, polyglutamates thereof, optical isomers thereof, particularly optically pure natural isomers thereof, and also mixtures of optical isomers also, particularly racemic mixtures, as well as pharmaceutically acceptable salts thereof also.

Folates are important cofactors in C1 transfer reactions, and are involved in key syntheses in human, animal and vegetable cells, particularly in DNA biosynthesis and in the methylation cycle. As drugs, folates have hitherto predominantly been used as the calcium salt of 5-formyl-5,6,7,8-tetrahydrofolic acid (leucovorin) or of 5-methyl-5,6,7,8-tetrahydrofolic acid (metafolin) for the treatment of megaloblastic folic acid anaemia, as an antidote for enhancing the compatibility of folic acid antagonists, particularly of aminopterin and methotrexate in cancer therapy (“antifolate rescue”), for enhancing the therapeutic effect of fluorinated pyrimidines and for the treatment of auto-immune diseases such as psoriasis, for enhancing the compatibility of certain anti-parasitic substances, for instance trimethoprim-sulfamethoxazole, and for reducing the toxicity of dideazatetrahydrofolates in chemotherapy.

Via pro-inflammatory cytokines, inflammations induce the synthesis of what are termed acute phase proteins. Despite the name, however, an acute phase response occurs not only in acute inflammatory processes but also in chronic inflammatory processes. Significantly increased circulatory acute phase parameters are found in infections, traumata, infarcts, arthritis and organ transplant rejection reactions, and in neoplasmas also. Moreover, cardio- and cerebrovascular diseases, and also adiposis, diabetes mellitus, uraemia, hypertonia, weight increase, hormone substitution, sleep disturbances, alcohol abuse, Alzheimer's disease or depression, auto-immune diseases and immunological disease formers commence with an enhanced acute phase response. Apart from the underlying diseases, therapeutic measures can also trigger an inflammation response, e.g. haemodialysis processes, lipidapheresis treatments, catheter dilatations or radiation therapy [Kushner I, Cleveland Clin J Med 2001; 68 (6): 535-37; Malle et al, Eur J Clin Invest 1996; 26: 427-35; Ridker et al, N Engl J Med 2000; 342: 836-43; Greaves et al, Trends in immunology 2002; 23 (11): 535-41; Wick et al, Trends in immunology 2001; 22 (12): 665-9]. The level of the inflammation markers reflects not only the presence but also the severity of the inflammation reaction, is of prognostic importance, and indicates the response in the course of therapy. In recent years, optimised test methods have emphasised the diagnostic value of acute phase markers for determining precisely the severity of chronic inflammation [Ridker P, Circulation 2001; 103 (13): 1813-18; Patel et al, Cleveland Clin J Med 2001; 68 (6): 521-34]. Arteriosclerosis in particular is increasingly being interpreted as an inflammatory disease, and increased levels of inflammation markers constitute significant factors of risk for cardio- and cerebrovascular occurrences [Ross R, N Engl J Med 1999; 340: 115-25, Ridker et al, N Engl J Med 1997; 336: 973-9, Haverkate et al, for the European Concerted Action on Thrombosis and Disabilities Angina Pectoris Study Group, The Lancet 1997; 349: 462-6; Ridker et al, N Engl J Med 2002; 347 (20): 1557-65].

C-reactive protein (CRP) is a protein which is formed in the liver and which is classified as a classical acute phase protein due to its rapid (within 12 hours) and extremely high (up to 2,000-fold) increase [Malle et al, Eur J Clin Invest 1996; 26: 427-35]. Functionally, it exhibits both pro- and anti-inflammatory properties. It binds penetrating extraneous substances, activates macrophages and the complement system, induces the release of cytokine and regulates leukocyte accumulation and adhesion [Patel et al, Cleveland Clin J Med 2001; 68: 521-34; Greaves et al, Trends in immunology 2002; 23 (11): 535-41]. In addition, current investigations have shown that CRP also has a direct pro-inflammatory effect on human endothelial cells [Pasceri et al, Circulation 2000; 102: 2165-8]. Primarily, it is associated with the inherent, non-specific immune response. The reference/normal value of CRP in plasma ranges up to 2 mg/l (adults and children), and different normal ranges are obtained depending on the test used and the group investigated.

Since the half-life of 24 hours is relatively short, changes in inflammatory occurrences are directly perceptible from the CRP concentration. CRP increases the most rapidly (over a few hours) and to the greatest extent for bacterial inflammation. For viral or local infections and chronic inflammation there is a lesser increase in CRP. On account of the very sensitive modern assays for CRP (high sensitive CRP), CRP is very suitable for observing the progress of inflammatory diseases [Roberts et al, Clin Chem 2001; 47: 418-25]. If antibiotic therapy is successful, the CRP level rapidly decreases again and if there is a lack of inflammation activity it exhibits a very slight variability between individuals, both diurnally and in the long term [Ockene et al, Clin Chem 2001; 47: 444-50]. Even slight increases in CRP without clinical indications of inflammation correlate with considerably increased cardio- and cerebrovascular morbidity and mortality [Ridker et al, N Engl J Med 1997; 336: 973-9; Haverkate et al, for the European Concerted Action on Thrombosis and Disabilities Angina Pectoris Study Group, The Lancet 1997; 349: 462-6; Harris et al, Am J Med 1999; 106: 506-12; Ridker et al, N Engl J Med 2002; 347 (20): 1557-65]. In this connection, aspirin also appears to develop its favourable effect by anti-inflammatory activity, and a slight increase in CRP is a significant marker for the necessity of this therapy [Ridker et al, N Engl J Med 1997; 336: 973-9]. The extent to which CRP is only a surrogate marker or is also an etiologically important factor is still unclear [Graeves et al, Trends in Immunology 2002; 23 (11): 535-41]. The CRP-reducing effect of statins, which has been briefly described, provides support for the key role of inflammation in arteriosclerosis, and according to these current studies the reduction of even a minimally increased CRP level is of comparable importance to the reduction of cholesterol [Albert et al, for the PRINCE Investigators, JAMA 2001; 286 (1): 64-70; Ridker et al, N Engl J Med 2001; 344 (26): 1959-65].

Increases of up to 10-100 mg/l are exhibited by slight to moderate, generally acute inflammatory processes or those of a restricted extent. These include local bacterial infection, uncomplicated cystitis, bronchitis, trauma, postoperative inflammation reactions, accidents, myocardial infarct, tuberculosis or sarcoidosis. Values of 100 mg/l or more in cases of acute diseases indicate a high or extended level of inflammation activity. These include sepsis, larger traumata, bacterial infections, metastasing tumours, active rheumatoid arthritis, seronegative spondylarthritis, immunovasculitis, polymyalgia rheumatica, Crohn's disease and deep vein thrombosis.

Serum amyloid A (SAA) protein consists of a family of polymorphous apolypoproteins which are mainly synthesised in the liver. SM is a very sensitive marker of the acute phase response and reacts to inflammation, necrosis and rejection reactions, and to the seeding of tumours. SM is an α1-globulin consisting of a simple polypeptide chain with a molecular weight between 11,500 and 14,000 daltons, and circulates in the blood bound to HDL [Malle et al, Eur J Clin Invest 1996; 26: 427-35].

The reference/normal value of SM in plasma ranges up to 1 mg/l. When there is inflammation, the SM concentration increases within a few hours to values of up to 2000 mg/l. As a rule, CRP and SM values run in parallel, although SM appears to react somewhat earlier and more dynamically and also increases more than CRP does [Gabay et al, N Engl J Med 1999; 340: 448-54; Liuzzo et al, N Engl J Med.1994; 331: 417-24; Wilkins et al, Clin Chem 1994; 40: 1284-90; Malle et al, Eur J Clin Invest 1996; 26: 427-35].

Increased CRP and SM values are associated with a whole series of diseases, particularly with inflammation and with diseases associated with inflammation, such as acute inflammatory, necrotising and tumour-like diseases, acute tissue lesions, bacterial and viral infections, rheumatic diseases such as rheumatoid arthritis, polyarthritis, spondylarthritis ankylopoetica, meningitis, pneumonia, pyelonephritis, acute bronchitis, tuberculosis, sepsis and acute pancreatitis, Alzheimer's disease, post-operative complications, rheumatic diseases, malignant tumours, rejection reactions, acute coronary thromboses, Reiter's syndrome, arthropathia psoriatica, colitis ulcerosa, Crohn's disease, etc. Furthermore, cardio- and cerebrovascular diseases such as adiposis, diabetes mellitus, uraemia, hypertonia, excessive body weight, hormone substitutions, sleep disturbances, alcohol abuse, Alzheimer's disease, anaemia or depression, organ transplants, auto-immune diseases and immunological diseases can set in with an increased acute phase response and correspondingly increased SM and CRP values. Moreover, increases in these inflammation markers can occur which are still in the region of the normal value, but which despite this set in with an increased risk of complications, and which can be seen as an indication for future therapy. Increases of this type also occur with the process which is described by the term “inflamm-aging” and which comprises an increase in inflammation burden in parallel with the ageing process [Kushner I, Cleveland Clin J Med 2001; 68 (6): 535-37; Malle et al, Eur J Clin Invest 1996; 26: 427-35; Ridker et al, N Engl J Med 2000; 342: 836-43; Neumann et al, Pteridines 1998; 9: 113-21; Muller T F, Papst Science Publ., Lengerich 1999, 175 pp].

The use of folates for producing a preparation suitable for the prevention or treatment of inflammation and of diseases associated with inflammation, particularly for influencing the levels of the inflammation markers CRP and SM, has not been proposed or described hitherto.

It has now surprisingly been found that the use of preparations containing folates is suitable for the treatment and prevention of inflammation and of diseases associated with inflammation, particularly for influencing the levels of the inflammation markers CRP and SM.

The folates which can be used include both pteroic acid monoglutamate (folic acid) and reduced forms such as dihydrofolates and tetrahydrofolates, polyglutamates thereof, optical isomers thereof and pharmaceutically acceptable salts thereof. The folates which are preferably used are tetrahydrofolates, particularly natural stereoisomeric forms of tetrahydrofolates such as 5-formyl-(6S)-tetrahydrofolic acid, 5-methyl-(6S)-tetrahydrofolic acid, 5,10-methylene-(6R)-tetrahydrofolic acid, 5,10-methenyl-(6R)-tetrahydrofolic acid, 10-formyl-(6R)-tetrahydrofolic acid, 5-formimino-(6S)-tetrahydrofolic acid or (6S)-tetrahydrofolic acid or pharmaceutically acceptable salts thereof. The folates which are used can generally be converted into one another by folate metabolism. 5-methyl-(6S)-tetrahydrofolic acid, 5-formyl-(6S)-tetrahydrofolic acid and pharmaceutically acceptable salts thereof are preferably used, however.

Pharmaceutically acceptable salts should be both pharmacologically acceptable and pharmaceutically acceptable. Pharmacologically and pharmaceutically acceptable salts such as these can be alkali metal or alkaline earth metal salts, preferably sodium, potassium, magnesium or calcium salts. The preparations relate to enteral (e.g. oral, sublingual or rectal), parenteral or topical (e.g. transdermal) forms. Organic or inorganic substances which do not react with the active ingredient can be used as carriers, e.g. water, oil, benzyl alcohol, polyethylene glycol, glycerol triacetate or other fatty acid glycerides, gelatine, lecithin, cyclodextrin, carbohydrates such as lactobiose or starch, magnesium stearate, talc or cellulose. Tablets, dragees, capsules, powder, syrup, concentrates or drops are preferably used for oral application, suppositories are preferably used for rectal application, and water- or oil-based solutions or lyophilisates are preferably used for parenteral application. Suspensions, emulsions or implants can also be used, and patches or creams can be used for topical application.

Preparations for parenteral application comprise sterile aqueous and nonaqueous injection solutions of the active compounds, which are preferably isotonic with the blood of the recipient.

These preparations can comprise stabilisers, additives for the controlled release of the pharmaceutically active compound, antioxidants, buffers, bacteriostatic agents and adjuvants for obtaining an isotonic solution. Aqueous and nonaqueous sterile suspensions can comprise suspension additives and thickeners. The preparation can exist as a single dose container or as a multiple dose container, e.g. as welded ampoules; it can be stored as a freeze- dried (lyophilised) product and when needed can be prepared for use by adding a sterile liquid, for example water or salt solution. Sterile powders, granules or tablets can be used similarly. All the preparations can additionally contain one or more active compounds which act separately or synergistically. In particular, these are substances which play a part in the folate cycle or which influence the folate cycle or which have an additional anti-inflammatory effect, such as vitamins, antioxidants such as vitamin E or beta carotene, radical scavengers, biopterins and/or other active ingredients. Examples include vitamin B₂, B₆, B₁₂ or vitamin C, glutathione, acetylcysteine, betaine, biopterins in all stages of oxidation, and isomeric forms of biopterin, especially L-erythro-biopterin, 7,8-dihydrobiopterin and 5,6,7,8-tetrahydrobiopterin, particularly L-sepiapterin, D-neopterin, xanthopterin and 6-hydroxymethyl- pterin. These substances additionally include lipid reducers such as clofibric acid derivatives (fibrates), e.g. clofibrate, bezafibrate, etofibrate, fenofibrate), ion exchange resins e.g. colestyramine or colestipol, Nicotinic acid (and derivatives thereof), e.g. acipimox, sitosterin and HMG-CoA-reductase inhibitors, e.g. atorvastatin, lovastatin, pravastatin, simvastatin, fluvastatin or cerivastatin. Another group of this class of substances comprises immuno-suppressive agents such as corticosteroids, mycophenolates, mofetil, rapamycin, calcineurin inhibitors, mono- and polyclonal antibodies, and growth factors such as erythropoetin or GM-CSF. A further group of this class of substances includes non-steroidal anti-inflammatory substances such as pentoxyfyllin, sulfasalazin, gold, aspirin, omega-3 fatty acids, thrombocyte aggregation inhibitors such as glycoprotein Ilb/IIa receptor inhibitors, hormones, flavinoids or other non-steroidal anti-inflammatory carboxylic acids such as aspirin, salsalate, diflunisal or choline magnesium trisalicylic acid, or other non-steroidal anti-inflammatory propionic acids such as ibuprofen, naproxen, fenoprofen, ketoprofen, flurbiprofen or oxaprozin, or other non-steroidal anti-inflammatory acetic acid derivatives such as indomethacin, tolmetin, sulindac, diclofenac or etodolac, or other non-steroidal anti-inflammatory fenamates such as meclofenamate or mefenamic acid, or other non-steroidal anti-inflammatory enolic acid derivatives such as piroxicam or phenylbutazone, or other non-steroidal anti-inflammatory naphthylkanones such as nabumetone, as well as COX-2 inhibitors such as celecoxib or rofecoxib. This class of substances also includes substances with an anti-inflammatory effect such as beta-blockers, anti-cytokine antibodies e.g. anti-TNF-alpha antibody, or perfusion solutions for organ preservation such as Eurocollins, HTK or University of Wisconsin (UW) solution.

The preparation comprises between 0.001 mg and 1,000 mg of the active ingredient per dose. In prophylaxis, preparations are used which preferably contain between 5 μg and 1,000 μg of the active ingredient per dose. In therapy, preparations are used which preferably contain between 0.1 mg and 200 mg of the active ingredient per dose. The dosage depends on the form of therapy, on the form of application of the preparation, and on the age, weight, nutrition and state of the patient. Treatment can commence with a lower dosage below the optimum amount and can be increased in order to achieve the optimum effect. The dosages used in prophylaxis preferably range between 5 μg and 5,000 μg per day, particularly between 100 μg and 1,000 μg per day. The optimum dosages in therapy range between 0.1 mg and 100 mg per day, particularly between 0.5 mg and 5 mg per day. Administration can be effected either as a single administration or as a repeated dose.

The preparations can be used for the prevention and treatment of inflammation and of diseases associated with inflammation in humans and in animals also.

Based on the preceding description, the person skilled in this field can immediately deduce the crucial elements of the invention, and, without departing from the basic idea and scope of the invention, can make changes and additions and can thereby adapt the invention to different requirements and conditions.

The entire disclosure of all patent applications, patents and publications which are cited in this text are included by reference thereto. The following examples can be carried out with similar success by replacing the generic or specifically described products and/or process conditions of this invention by those which are given in the following examples. The following specific embodiments are also purely exemplary, and should by no means be considered as limiting the remainder of the disclosure.

### EXAMPLES TO ILLUSTRATE THE INVENTION

### Example 1

### A Tablet Containing 1 mg 5-formyl-(6S)-tetrahydrofolic Acid

A mixture of 13.3 g 5-formyl-(6S)-tetrahydrofolic acid, calcium salt pentahydrate (corresponding to 10 g 5-formyl-(6S)-tetrahydrofolic acid), 4 kg lactose, 1.2 kg starch, 0.2 kg talc and 0.1 kg magnesium stearate was pressed to form tablets, so that each tablet contained 1 mg 5-formyl-(6S)-tetrahydrofolic acid.

The tablet could be used coated, as a film tablet, or ground and introduced into capsules.

### Example 2

### Suppositories Containing 60 mg 5-methyl-(6S)-tetrahydrofolic Acid

A mixture of 632 g 5-methyl-(6S)-tetrahydrofolic acid, calcium salt pentahydrate (corresponding to 500 g 5-methyl-(6S)-tetrahydrofolic acid), 50 g hydroxypropyl cellulose and 2 kg semi-synthetic glyceride was melted to produce suppositories, so that each suppository contained 500 mg 5-methyl-(6S)-tetrahydrofolic acid.

### Example 3

### An Injection Solution Containing 0.5 mg 5-methyl-(6S)-tetrahydrofolic Acid

0.5 g 5-methyl-(6S)-tetrahydrofolic acid, 10 g glutathione, 30 g citric acid, 160 g mannitol, 1 g methyl-p-hydroxybenzoic acid, 17.7 g sodium hydroxide (or the requisite amount to adjust the pH of the solution to between 7.3 and 7.8) were dissolved in 3 litres water for injection and were introduced into ampoules so that each ampoule contained 0.5 mg 5-methyl-(6S)-tetrahydrofolic acid.

### Example 4

### An Injectable Lyophilisate Containing 1 mg (6S)-tetrahydrofolic Acid

A solution of 1 g (6S)-tetrahydrofolic acid, sodium salt in 1,000 ml double distilled water was filtered under sterile conditions into ampoules and lyophilised so that each ampoule contained 1 mg (6S)-tetrahydrofolic acid.

Tetrahydrofolic acid is very sensitive to oxygen, and therefore has to be handled under conditions which are strictly oxygen-free. The use of an antioxidant such as ascorbic acid may be necessary.

### Example 5

### An Injectable Lyophilisate Containing 20 mg 5,10-methylene-(6R)-tetrahydrofolic Acid

A solution of 10 g of a 0-hydroxypropyl-cyclodextrin inclusion compound of 5,10-methylene-(6R)-tetrahydrofolic acid, sodium salt in 2,000 ml double distilled water was filtered under sterile conditions into ampoules so that each ampoule contained 20 mg 5,10-methylene-(6R)-tetrahydrofolic acid.

5,10-methylenetetrahydrofolic acid necessitates the same precautionary measures as those used for tetrahydrofolic acid (Example 4).

### Example 6

### A Tablet Containing 0.4 mg 5-formyl-(6S)-tetrahydrofolic Acid

A mixture of 5.32 g 5-formyl-(6S)-tetrahydrofolic acid, calcium salt pentahydrate (corresponding to 4 g 5-formyl-(6S)-tetrahydrofolic acid), 4 kg lactose, 1.2 kg starch, 0.2 kg talc and 0.1 kg magnesium stearate was pressed to form tablets, so that each tablet contained 4 mg 5-formyl-(6S)-tetrahydrofolic acid.

The tablet could be used coated, as a film tablet, or ground and introduced into capsules.

### Example 7

### An Injectable Lyophilisate Containing 100 μg 5-methyl-(6S)-tetrahydrofolic Acid

A solution of 100 mg 5-methyl-(6S)-tetrahydrofolic acid, sodium salt in 1,000 ml double distilled water was filtered under sterile conditions and under a protective gas into ampoules, and was lyophilised, so that each ampoule contained 100 μg 5-methyl-(6S)-tetrahydrofolic acid.

Tetrahydrofolic acid is very sensitive to oxygen, and therefore has to be handled under conditions which are strictly oxygen-free. The use of an antioxidant such as ascorbic acid may be necessary.

### Example 8

### A tablet containing 15 mg 5-methyl-(6S)-tetrahydrofolic Acid

A mixture of 19.18 g 5-methyl-(6S)-tetrahydrofolic acid, calcium salt pentahydrate (corresponding to 15 g 5-methyl-(6S)-tetrahydrofolic acid), 120 g lactose, 21.5 g maize starch, 7.08 g acetyl cellulose, 2.28 g diethyl phthalate, 0.64 g silicone HK-15 and 2 g magnesium stearate was pressed to form tablets, so that each tablet contained 15 mg 5-methyl-(6S)-tetrahydrofolic acid.

The tablet could be used coated, as a film tablet, or ground and introduced into capsules.

### Example 9

### Tablets Containing 15 mg 5-methyl-(6R,S)-tetrahydrofolic Acid

Using an analogous procedure to that described in Example 8, tablets were produced which contained 15 mg 5-methyl-(6R,S)-tetrahydrofolic acid with maize starch, lactose, magnesium stearate, polyethylene glycol 6000, polymethacrylate, polysorbitol 80, dimethylpolysiloxane, sodium hydroxide and talc.

### Example 10

### Tablets Containing 15 mg 5-formyl-(6R,S)-tetrahydrofolic Acid

Using an analogous procedure to that described in Example 8, tablets were produced which contained 15 mg 5-formyl-(6R,S)-tetrahydrofolic acid with maize starch, lactose, magnesium stearate, polyethylene glycol 6000, polymethacrylate, polysorbitol 80, dimethylpolysiloxane, sodium hydroxide and talc.

### Example 11

### A Combination Preparation Comprising 5-methyl-(6S)-tetrahydrofolic Acid, Vitamin B₆ and Vitamin B₁₂

For preparations for oral application, a film tablet was formulated which contained the following constituents:
10 mg 5-methyl-(6S)-tetrahydrofolic acid
100 mg vitamin B₆
1 mg vitamin B₁₂ pharmaceutically acceptable adjuvants

The combination preparation could also be formulated as a solution, e.g. for parenteral application.

### Example 12

### A Basic Vitamin Preparation Containing 5-methyl-(6S)-tetrahydrofolic Acid and Other Ingredients

For preparations for oral application, a film tablet was formulated which contained the following constituents:
0.4 mg 5-methyl-(6S)-tetrahydrofolic acid
3 mg vitamin B₁
1.7 mg vitamin B₂
10 mg vitamin B₆
0.006 mg vitamin B₁₂
60 mg vitamin C
0.3 mg biotin
20 mg nicotinamide
10 mg pantothenic acid pharmaceutically acceptable adjuvants

The combination preparation could also be formulated as a solution, e.g. for parenteral application.

### Example 13

### A Combination Preparation Containing 5-methyl-(6S)-tetrahydrofolic Acid and Betaine Amongst Other Ingredients

A combination preparation was produced analogously to Examples 11 and 12, and in addition to the amount of 5-methyl-(6S)-tetrahydrofolic acid which is customary for the corresponding application also contained the amount of betaine which is customary for this application.

### Example 14

### A Combination Preparation Containing 5-methyl-(6S)-tetrahydrofolic Acid and Tetrahydrobiopterin Amongst Other Ingredients

A combination preparation was produced analogously to Examples 11 and 12, and in addition to the amount of 5-methyl-(6S)-tetrahydrofolic acid which is customary for the corresponding application also contained the amount of tetrahydrobiopterin which is customary for this application.

### Example 15

### A Combination Preparation Containing 5-methyl-(6S)-tetrahydrofolic Acid and Statins Amongst Other Ingredients

A combination preparation was produced analogously to Examples 11 and 12, and in addition to the amount of 5-methyl-(6S)-tetrahydrofolic acid which is customary for the corresponding application also contained the amount of statins, such as atorvastatin, lovastatin, pravastatin, simvastatin, fluvastatin or cerivastatin, which is customary for this application.

### Example 16

### A Combination Preparation Containing 5-methyl-(6S)-tetrahydrofolic Acid and Aspirin Amongst Other Ingredients

A combination preparation was produced analogously to Examples 11 and 12, and in addition to the amount of 5-methyl-(6S)-tetrahydrofolic acid which is customary for the corresponding application also contained the amount of aspirin which is customary for this application.

### Example 17

### A Combination Preparation Containing 5-methyl-(6S)-tetrahydrofolic Acid and Additional Active Ingredients

A combination preparation was produced analogously to Examples 11 and 12, and in addition to the amount of 5-methyl-(6S)-tetrahydrofolic acid which is customary for the corresponding application also contained the amount which is customary for this application of vitamin B₂, vitamin B₆, vitamin B₁₂ or vitamin C, gluthatione, acetylcysteine, pentoxifyllin, omega-3 fatty acids, vitamin E, thrombocyte aggregation inhibitors such as glycoprotein IIb/IIIa receptor inhibitors, beta blockers, hormones, flavinoids or other non-steroidal anti- inflammatory substances such as ibuprofen, indomethacin, diclofenac, piroxicam, COX-2 inhibitors or immunosuppressive agents, perfusion solutions or antibodies with an anti-inflammatory effect.

Determination of Clinical Data

Clinical data were obtained via the prospective, randomised double blind study described below.

141 patients with terminal renal insufficiency, who were being treated with chronic haemodialysis on 3 days each week, and who were between 24 and 90 years old, were divided randomly into 4 therapy groups according to the sex of the patient and the presence or absence of a C677T point mutation on the gene for methylene tetrahydrofolate reductase. Randomisation based on the methylene tetrahydrofolate reductase mutation was effected in order to prevent an imbalance occurring within the individual therapy groups with regard to enzyme activity and thus with regard to folic acid metabolism also.

All the patients received a film tablet with the following composition daily, as a basic vitamin supplement:

| | | |
|---|---|---|
| vitamin B₁ | 3 | mg |
| vitamin B₂ | 1.7 | mg |
| vitamin B₆ | 10 | mg |
| vitamin B₁₂ | 6 | μg |
| vitamin C | 60 | mg |
| biotin | 0.3 | mg |
| folic acid | 1 | mg |
| nicotinamide | 20 | mg |
| pantothenic acid | 10 | mg |

Vitamins B₆ (pyridoxine) and B₁₂ (cobalamin) play an important part as cofactors in folic acid metabolism. They were therefore added as supplements, firstly to increase the efficacy of the folates and secondly to prevent neurological damage due to vitamin B₁₂ deficiency [Bostom et al, Kidney Int 1997; 52: 10-20; Homocysteine lowering trialists' collaboration, BMJ 1998; 316: 894-8, Bostom et al, Circulation 2000; 101: 2829-32].

In all the patients in the study, a uniform vitamin status was achieved even at the start of the study, and a pronounced vitamin deficiency state was avoided, by means of the aforementioned basic vitamin substitution.

As is illustrated graphically below, the patients were given the following substances for a total of 6 weeks in addition to the already existing basic vitamin preparation:

| | |
|---|---|
| PGA group | 7.5 mg folic acid per day |
| FTHF group | 15 mg 5-formyl-(6R,S)-tetrahydrofolic acid per day |
| MTHF group | 15 mg 5-methyl-(6R,S)-tetrahydrofolic acid per day |
| PLAC group | placebo |

[image]

The first blood sample in the study was taken before the commencement of the administration of additional vitamins. The second blood sample was taken 3 weeks after the start and the third at the end of the study, i.e. after 6 weeks.

The following measured quantities were each determined from plasma samples from the 3 blood samples taken:
homocysteine (total) (Hcys)
total folate (Fol)
vitamin B₁₂ (B12)
vitamin B₆ (B6)
neopterin (NEOP)
creatinine (Krea)
LDL- and HDL-cholesterol (HDL-Chol, LDL-Chol)
triglyceride (TG)
C-reactive protein (CRP)
amyloid A protein (SAA)

The parameters homocysteine, total folate and vitamin B₆ and B₁₂ were determined in the plasma of the patients. On account of the known association between arteriosclerosis and lipid metabolism, the triglyceride and cholesterol fractions LDL and HDL were also measured. In addition, neopterin was measured in the plasma samples as a parameter of an immune response. CRP and SAA were determined as markers for an inflammatory response, as mentioned above.

Each blood sample was taken at the commencement of dialysis treatment, via the horizontal dialysis needle. Each time a blood sample was taken, a total of 30 ml full blood was removed for the investigations.

Results of the Clinical Study

The results of the study with respect to the inflammatory markers CRP and SAA are presented below.

The drop-out rate was 14.9%, i.e. of the total of 141 patients, who were included in the randomisation, 121 completed the study. All the readings obtained were taken into account in the evaluation.

Apart from descriptive statistics, the parametric test procedures ANOVA (analysis of variance), Student's t-test, and correlation and “matched pair” analyses were performed using the JMP-SAS statistics package.

Distribution of the Basic Characteristic Values

Table 1 below is a summary of the significant values obtained in the laboratory, and of the distribution, mean and range or standard deviation thereof before the start of folate therapy.

TABLE 1

| | | | | | | Sample > |
|---|---|---|---|---|---|---|
| Group | Total | FTHF | MTHF | PGA | PLAC | F³ |
| Number [n] | 141 | 37 | 35 | 36 | 33 | |
| Women [n] | 57 | 14 | 14 | 14 | 15 | |
| Old persons | 64 | 60 | 68 | 67 | 60 | |
| [a]¹ | 24-90 | 24-89 | 44-90 | 43-87 | 37-80 | |
| Hcys [μmol/ | 28.8 | 30.3 | 28.5 | 27.7 | 28.3 | 0.87 |
| l]² | 13.9 | 13.5 | 12.7 | 13.4 | 16.3 | |
| folate [nmol/ | 75.2 | 62.3 | 80.9 | 84.6 | 72.6 | 0.53 |
| l]² | 68.5 | 64.8 | 58.4 | 93.2 | 46.7 | |
| CRP [mg/l]² | 14.1 | 16.3 | 18.9 | 9.6 | 11.3 | 0.37 |
| | 24.7 | 38.4 | 21.9 | 8.6 | 18.2 | |
| SAA [mg/l]² | 23.9 | 28.4 | 34.5 | 11.2 | 21.3 | 0.56 |
| | 71.7 | 103 | 72.7 | 15.5 | 65.2 | |
| NEOP | 185 | 162 | 174 | 238 | 164 | 0.28 |
| [μmol/mol | 185 | 111 | 95 | 326 | 88 | |
| crea]² | | | | | | |

¹mean and range
²mean and standard deviation
³ANOVA

Both in the ANOVA and in the Student's t Test there were no significant differences between the individual therapy groups for any of the parameters illustrated. Randomisation was therefore successful.

Level of Acute Phase Proteins in the Dialysis Patients Investigated

CRP and SAA were both measured by means of immuno-nephelometry and tests supplied by DadeBehring (N High Sensitivity CRP and N Latex SAA assays). The reference values determined for healthy persons for the tests used are:

CRP—mean 1.6 mg/l, median 1.1 mg/l, 95% percentile 5 mg/l

SAA—mean 2.6 mg/l, median 2.0 mg/l, 95% percentile 6.8 mg/l.

On average, the dialysis patients investigated exhibited SAA or CRP values which were increased about 10-fold. 212/PCT

Correlations Between the Individual Parameters

A correlation analysis for the initial values of CRP and SM gave the values presented in Table 2 below.

TABLE 2

| | Pearson | p value |
|---|---|---|
| SAA vs. CRP | 0.912 | *** |

*** i.e. p value < 0.001

As shown, there is a highly significant correlation for the acute phase proteins SAA and CRP. However, there was no correlation between the initial values of neopterin and CRP or SM. There was just as little correlation between the homocysteine and folate values before therapy and the inflammation markers (data not presented).

Effects of Folate Therapy on the Parameters

The effects of folate therapy are firstly presented below as a histogram.

FIGS. 1 and 2 show, as bar histograms, the mean values and standard deviations of the parameters investigated for the different therapy groups at the start of the study (BL baseline), after 3 weeks (3 w) and after 6 weeks (6 w) of folate therapy. In addition, the median values over the course of therapy are shown graphically in FIGS. 3 and 4. The measured values are additionally presented in tabular form in Table 3.

[image]

[image]

[image]

[image]

TABLE 3

| | Total | | | FTHF | | | MTHF | | | PGA | | | PLAC | | |
|---|---|---|---|---|---|---|---|---|---|---|---|---|---|---|---|
| | BL | 3 w | 6 w | BL | 3 w | 6 w | BL | 3 w | 6 w | BL | 3 w | 6 w | BL | 3 w | 6 w |
| CRP[n] | 139 | 114 | 119 | 37 | 28 | 30 | 35 | 29 | 30 | 36 | 31 | 31 | 31 | 26 | 28 |
| MW | 14 | 10 | 11 | 16 | 9 | 18 | 19 | 11 | 9 | 10 | 10 | 9 | 11 | 9 | 10 |
| SD | 25 | 10 | 22 | 38 | 11 | 41 | 22 | 9 | 7 | 9 | 9 | 9 | 18 | 9 | 10 |
| Median | 8 | 8 | 7 | 9 | 5 | 6 | 11 | 8 | 7 | 7 | 8 | 6 | 6 | 7 | 6 |
| SAA[n] | 139 | 118 | 115 | 37 | 30 | 30 | 35 | 30 | 29 | 36 | 32 | 29 | 31 | 26 | 27 |
| MW | 24 | 14 | 19 | 28 | 8 | 35 | 34 | 19 | 13 | 11 | 12 | 13 | 21 | 15 | 13 |
| SD | 72 | 24 | 55 | 103 | 12 | 104 | 73 | 27 | 11 | 15 | 28 | 15 | 65 | 25 | 12 |
| Median | 7 | 7 | 8 | 6 | 4 | 6 | 11 | 10 | 9 | 7 | 6 | 10 | 6 | 8 | 8 |

For the patients for whom all the measured values could be ascertained completely at any time, a “matched pair” analysis was performed in addition. For the individual patient, the values before therapy were compared, pair-wise, with those after 3 and 6 weeks of therapy. Table 4 gives the differences determined for the inflammatory markers and for homocysteine (HCYS).

TABLE 4

| | Total⁴ | | | FTHF | | | MTHF | | | PGA | | | PLAC | | |
|---|---|---|---|---|---|---|---|---|---|---|---|---|---|---|---|
| | ¹ | ² | ³ | ¹ | ² | ³ | ¹ | ² | ³ | ¹ | ² | ³ | ¹ | ² | ³ |
| Hcys | ★★★ | ★★★ | Ø | ★ | ★★ | Ø | Ø | ★ | Ø | ★ | ★ | Ø | Ø | Ø | Ø |
| CRP | ★★ | ★★ | Ø | ★ | Ø | Ø | ★ | ★★ | ★ | Ø | Ø | Ø | Ø | Ø | Ø |
| SAA | ★ | Ø | Ø | ★ | Ø | ★ | Ø | Ø | Ø | Ø | Ø | ★ | Ø | Ø | Ø |

¹ BL vs 3 w, comparison between initial value and value after 3 weeks
² BL vs 6 w, comparison between initial value and value after 6 weeks
³ 3 w vs 6 w, comparison between value after 3 weeks and value after 6 weeks
⁴ Total, i.e. all the data except for the values of the placebo group
★ i.e. p < 0.05, ★★ i.e. p < 0.001, ★★★ i.e. p < 0.0001

As is also illustrated in FIGS. 1 to 4, the inflammation markers CRP and SM exhibit a decrease during folate therapy which is sometimes significant. The reduced folates appear to achieve a stronger effect. CRP exhibits the most significant reaction. In the placebo group, CRP and SAA did not vary over the course of the study.

Conclusions from the Clinical Study

Patients with chronic renal insufficiency who are receiving haemodialysis treatment have an increased inflammatory burden which is characterised, as confirmed in the present study, by what are clearly pathological CRP and SAA values. It was not possible to establish a correlation with homocysteine values, which are likewise increased, and it accordingly appears that two independent processes are in operation.

Therapy with folates induced a trend, which was sometimes even statistically significant, towards a decrease of the CRP level in particular. Patients treated with 5-methyl-(6R,S)-tetahydrofolic acid exhibited the most pronounced decrease in inflammation markers.

The effect of folates on acute phase parameters is all the more surprising since it was possible to observe trends and sometimes even significant effects for the small number of patients and over the very brief duration of the study and thus of therapy.

Similarly to statins, folates, particularly reduced folates, especially MTHF, reduce the acute phase response as read from CRP and SAA levels, and therefore have an anti-inflammatory effect—acute and chronic.

1. The use of folates for producing a pharmaceutical preparation suitable for the prevention and treatment of inflammation and of diseases associated with inflammation.

2. The use of folates for producing a pharmaceutical preparation suitable for influencing CRP and/or SAA levels.

3. A use of folates according to claim 1 characterised in that pteroic acid monoglutamate (folic acid), dihydrofolic acid, 5-formyltetrahydrofolic acid, 5-methyltetrahydrofolic acid, 5,10-methylenetetrahydrofolic acid, 5,10-methenyltetrahydrofolic acid, 10-formyltetrahydrofolic acid or tetrahydrofolic acid, polyglutamates thereof, optical isomers thereof, particularly optically pure natural isomers thereof, and mixtures of optical isomers also, particularly racemic mixtures, as well as pharmaceutically acceptable salts thereof also, are used as a folate.

4. A use of folates according to claim 1, characterised in that 5-methyl-(6S)-tetrahydrofolic acid, 5-methyl-(6R,S)-tetrahydrofolic acid, 5-formyl-(6S)-tetrahydrofolic acid or 5-formyl-(6R,S)-tetrahydrofolic acid, or a pharmaceutically acceptable salt thereof, is used as a folate.

5. A use of folates according to claim 1, characterised in that 5-methyl-(6S)-tetrahydrofolic acid or 5-methyl-(6R,S)-tetrahydrofolic acid, or a pharmaceutically acceptable salt of 5- methyl-(6S)-tetrahydrofolic acid or 5-methyl-(6R,S)-tetrahydrofolic acid is used as a folate and is administered for methylene tetrahydrofolate reductase anomaly.

6. A method for the treatment and/or prophylaxis of inflammation and of diseases associated with inflammation, characterised in that at least one folate or a pharmaceutically acceptable salt of a folate is used for a time and under conditions sufficient for reducing, for retarding an increase in or for otherwise influencing CRP or SAA levels, or the levels of a derivative or homologue.

7. A pharmaceutical composition for reducing, for retarding an increase in or for otherwise influencing inflammation and diseases associated with inflammation by impeding, inhibiting or by otherwise reducing CRP or SAA levels or the levels of a derivative or homologue, characterised in that it comprises at least one folate or a pharmaceutically acceptable salt of a folate as an active ingredient.

8. A pharmaceutical composition for influencing CRP and/or SAA levels, characterised in that as an active ingredient it comprises at least one compound which is selected is from the group consisting of pteroic acid monoglutamate (folic acid), dihydrofolic acid, 5-formyltetrahydrofolic acid, 5-methyltetrahydrofolic acid, 5,10-methylenetetrahydrofolic acid, 5,10-methenyltetrahydrofolic acid, 10-formyltetrahydrofolic acid or tetrahydrofolic acid, polyglutamates thereof, optical isomers thereof, particularly optically pure natural isomers thereof, and mixtures of optical isomers also, particularly racemic mixtures, as well as pharmaceutically acceptable salts thereof also, together with pharmaceutically acceptable active ingredients and adjuvants.

9. A pharmaceutical composition for influencing CRP and/or SAA levels in the presence of methylene tetrahydrofolate reductase anomaly, characterised in that as an active ingredient it comprises 5-methyl-(6S)-tetrahydrofolic acid or 5-methyl-(6R,S)-tetrahydrofolic acid, or a pharmaceutically acceptable salt of 5-methyl-(6S)-tetrahydrofolic acid or 5-methyl-(6R,S)-tetrahydrofolic acid, together with pharmaceutically acceptable active ingredients and adjuvants.

10. A pharmaceutical composition according to claim 7, additionally comprising at least one vitamin from the B group.

11. A pharmaceutical composition according to claim 10, characterised in that it comprises vitamin B₂, B₆ and/or B₁₂ as a vitamin from the B group.

12. A pharmaceutical composition according to claim 7, additionally comprising at least one antioxidant or a radical scavenger.

13. A pharmaceutical composition according to claim 12, characterised in that it comprises vitamin C or reduced glutathione as an antioxidant or radical scavenger.

14. A pharmaceutical composition according to claim 7, comprising tetrahydrobiopterin as a further active ingredient in addition to folates.

15. A pharmaceutical composition according to claim 7, comprising omega-3 fatty acids as a further active ingredient in addition to folates.

16. A pharmaceutical composition according to claim 7, comprising at least one further active ingredient in addition to folates.

17. A pharmaceutical composition according to claim 16, characterised in that as a further active ingredient it comprises statine, acetylcysteine, pentoxifyllin or aspirin.

18. A pharmaceutical composition according to claim 16, characterised in that as a further active ingredient it comprises betaine, pentoxifyllin, vitamin E, thrombocyte aggregation inhibitors such as glycoprotein IIb/IIIa receptor inhibitors, beta-blockers, hormones, flavinoids, lipid reducers or other non-steroid anti-inflammatory substances.
